(19) United States
(12) Patent Application Publication
(10) Pub. No.: US 2005/0080468 A1
(43) Pub. Date: Apr. 14, 2005

(54) Methods and apparatus for treating diseased tissue

(76) Inventor: Lawrence C. Christman, Bethany Beach, DE (US)

Correspondence Address: POTTER ANDERSON & CORROON LLP;ATTN: KATHLEEN W. GEIGER, ESQ., P.O. BOX 951, WILMINGTON, DE, 19899-0951, US

(21) Appl. No.: 10/473,712

(22) PCT Filed: Mar. 26, 2002
(86) PCT No.: PCT/US02/09035

Related U.S. Application Data
(60) Provisional application No. 60/278,604, filed on Mar. 26, 2001.

(51) Int. Cl. A61F 7/00; A61F 7/12

(52) U.S. Cl. 607/96; 600/2

(57) ABSTRACT

Methods and devices for treating diseased tissue using ultrasound waves are provided. The ultrasound waves are not focused, and advantageously are administered in conjunction with radiation therapy and/or chemotherapy. The methods and devices provide controlled delivery of ultrasound, and are particularly effective in treating cancers of non-bony tissue such as the breast.

### RELATED APPLICATION

This application claims the priority of U.S. provisional patent application Ser. No. 60/278,604, the disclosure of which is incorporated herein in its entirety.

### FIELD OF THE INVENTION

The invention relates to methods and an apparatus for treating diseased tissue at least partly by temperature elevation and/or regulation. In particular, the invention relates to methods and an apparatus for treating diseased tissue, using ultrasonic therapy for imparting energy to the tissue, optionally in conjunction with other forms of therapy that may be carried on simultaneously or sequentially, including radiation therapy and chemotherapy.

### BACKGROUND OF THE INVENTION

Ultrasonic waves are effective in providing therapeutic heating of tissue, which can be a useful technique in treating diseased tissue such as cancerous tissue. The use of therapeutic heating for treating diseased tissue relies on the effects of hyperthermia on tissue, which effects can be enhanced by the simultaneous application of radiation, among other ways.

Hyperthermia may be achieved by the application of energy in the form of, for example, microwaves, ultrasound waves, or radio-frequency waves. Hyperthermic toxicity, the direct killing of cells by overheating, is described in Overgaard, “The Current and Potential Role of Hyperthermia in Radiotherapy”, Int. J. Radiation Oncology Biol. Phys., Vol. 16, pp. 535-549 (1989).

When hyperthermia and radiation are applied to tissue, an effect known as “hyperthermic radiosensitization” occurs. Radiation and hyperthermic treatment may be applied either simultaneously or sequentially.

A need continues for new and/or improved methods and devices for using hyperthermia, and particularly hyperthermia generated by ultrasonic waves, in medical treatment applications. Methods that allow increased control of the level and duration of local heating are desired, as well as methods that provide effective treatment, while minimizing unnecessary exposure of patients to radiation and excessive damage to healthy cells, are also desired. The present invention is directed to these and other important ends.

### SUMMARY OF THE INVENTION

One aspect of the invention is a method for treating diseased tissue in a mammal. The method includes inducing regional hyperthermia in the tissue by applying unfocused ultrasound waves to the tissue so as to achieve a diffuse area of heating. The heating can be regulated and can be applied as a therapeutic regimen. In preferred embodiments the method is used to treat a human patient.

In one embodiment of the invention, the method further includes applying to the tissue high-energy radiation, such as ionizing radiation, preferably concentrated on a treatment zone. In some preferred embodiments, the unfocused ultrasound waves are applied in conjunction with the high-energy radiation.

In another preferred embodiment of the invention, the method further includes administering to the mammal one or more chemotherapeutic agents in conjunction with the other steps, either simultaneously or sequentially.

An inventive aspect is use of a particular device for providing ultrasound-generated hyperthermic treatment to a patient having diseased tissue in need of treatment. The device includes a means for generating unfocused ultrasound waves; a means for obtaining data from the tissue in situ; a central processing unit; an exposure tank; and a means for directing said ultrasound waves to said tissue. Optionally, the device can include an isolated chamber. The optional isolated chamber allows for coupling of acoustic energy to targeted tissue.

These and other aspects of the present invention will be apparent to one skilled in the art in view of the following disclosure and the appended claims.

### BRIEF DESCRIPTION OF THE DRAWINGS

The drawings show a number of exemplary or preferred arrangements for purposes of illustration. In the drawings,

FIG. 1 is an elevation view generally depicting a patient disposed for application of an ultrasound tissue heating treatment according to one exemplary application of the invention.

FIG. 2 is a detailed view from FIG. 1, partly in section, and depicting immersion of tissue to be treated, specifically at and adjacent to a human breast.

FIG. 3 is a partial section view as in FIG. 2, illustrating a brachytherapy application associated with temperature regulation according to an embodiment of the invention.

FIG. 4 is a section view as in FIG. 2, illustrating tissue heating using certain contact and non-contact shielding structures for modifying application of acoustic energy.

FIG. 5 is a corresponding section view illustrating insertion of a flexible cannula in association with a treatment step.

FIG. 6 is an elevation view illustrating application of the invention in association with a whole body hyperthermia step.

FIG. 7 is a schematic block diagram illustrating a simulation and control subsystem including a number of sources of input data or measurements and a number of associated control and informational outputs according to the invention, for effecting treatment as described herein.

FIG. 8 is a section view as in FIG. 2, illustrating the subsurface installation of temperature sensors, in particular miniature thermistors, for monitoring local temperature conditions in an area of tissue at or related to an area being subjected to treatment.

FIG. 9 is a schematic block diagram representing a state illustration of operational elements embodied in practicing the method of the invention.

FIG. 10 is a hardware block diagram illustrating a number of functional elements for use according to the method of the invention.

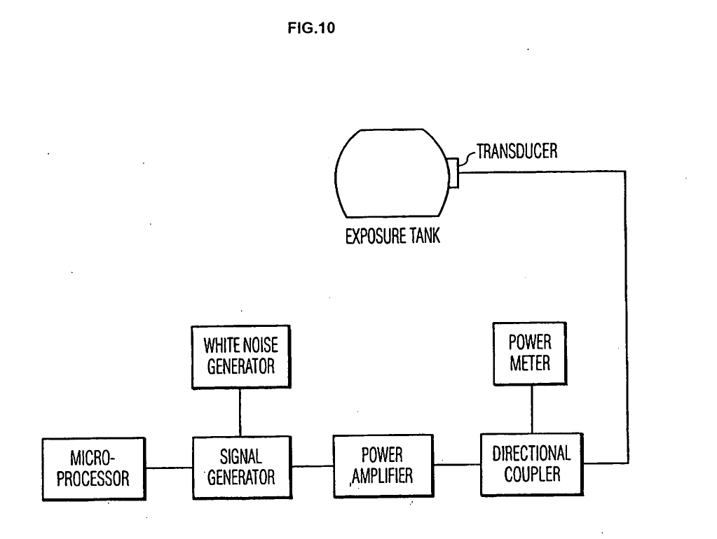

FIG. 11 is an elevation view illustrating application of the invention in treating the kidney.

### DETAILED DESCRIPTION OF PREFERRED EMBODIMENTS

The invention provides methods and devices for the treatment of diseased tissue, in particular cancerous tissue, by the application of ultrasonic waves to the tissue to generate hyperthermia in the tissue. It has now been discovered that the application of ultrasound waves to generate hyperthermia throughout targeted, diseased tissue and surrounding tissue, substantially without focusing the ultrasound waves, improves the effectiveness of hyperthermia in treating diseased tissue. The improved effectiveness has been observed in the treatment of cancer tumors, especially breast cancer tumors, and is enhanced when hyperthermia is used in conjunction with radiation and/or chemotherapy. It has further been found that the combination of chemotherapy and ultrasound-induced hyperthermia is more effective in treatment of cancerous tumors than chemotherapy alone or ultrasound-induced hyperthermia alone. While it is not intended that the present invention be bound by any particular theory, it is believed that hyperthermia generates localized increased perfusion in targeted and surrounding tissue, which improves the delivery of chemotherapy agents to targeted tissue such as tumors. It is also believed that hyperthermia and ultrasound can change the transport characteristics of cell membranes.

The desired cytotoxic effect of heat treatment on targeted cells is optimal after the stage at which angiogenesis occurs. Angiogenesis, the development of new blood vessels, is necessary to maintain homeostasis (normal levels of oxygen and nutrients) with growth of a tumor. As a result, hyperthermia, or a combination of hyperthermia and radiation, is commonly believed to be effective for treating tumors. The tumor cells may become hypoxic if the tumor has outgrown blood supply. Hyperthermia is particularly effective for treating hypoxic cells, while other treatment modalities including ionizing radiation may fail. Since the production of free radicals is less likely in hypoxic cells than in well-oxygenated cells, and free radicals are produced when radiation interacts with water in the presence of dissolved oxygen atoms, hypoxic cells are particularly resistant to injury from radiation. Since blood flow is restricted in hypoxic cells, such cells may also be resistant to chemotherapy, since they are likely to receive a lower local or internal dose of the drug. In contrast, perhaps due to other cytotoxic mechanisms, the present invention is particularly applicable to treatment of Stage I and Stage II breast cancer, and to provide treatment after a tumor has been removed, e.g., to eliminate microtumors and the like that can lead to re-occurrence. The present invention is not to be limited to any particular mechanistic theory, as these or other cytotoxic mechanisms may be involved in the effectiveness of the methods disclosed herein in treating cancer and particularly microtumors, which are present long before angiongenesis occurs.

The methods and devices of the present invention utilize diffuse ultrasonic waves. Ultrasonic waves can generally be classified as focused waves, plane waves, or diffuse waves. Diffuse ultrasound waves can be visualized as a superposition of a plurality of plane waves, which can have a variety of amplitudes, phases and/or propagation directions. Diffuse ultrasound comprises a continuum of diffuseness (from totally diffuse to less diffuse to nearly planar). One advantage of diffuse ultrasound is substantial elimination of the importance of the position of transducers, the position of the target tissue, and the position of sensors used to measure decay rate. Also the exposure field can be modeled because of the statistical nature of a diffuse field. At any point within the diffuse field over a sufficiently long period of time, the mean amplitude of all wave fronts is uniform and a wave has an equal probability of traveling in any direction. Moreover, the absorption of ultrasound waves by targeted tissue, which is a linear function of frequency for plane waves in mammalian soft tissue at the frequencies of interest, is not linear with diffuse ultrasound. Rather, for diffuse ultrasound, because the ultrasound energy approaches the target tissue from a plurality of angles, the absorption is approximately exponential near the tissue surface and becomes linear with deeper penetration. However, it will be recognized that in some applications, reduced diffuseness may be desired at the surface of the target tissue. Additionally, the diffuseness of the ultrasound may be reduced as the ultrasound travels through tissue. Thus, while diffuseness of ultrasound is advantageous, the degree of diffuseness may be less than 100%.

The therapeutic application of diffuse ultrasound waves to tissue substantially without focusing to generate hyperthermia may be referred to as “diffuse field ultrasound hyperthermia” or “DFHT”. “Substantially without focusing” and “substantially unfocused” mean that no external device is used to create a focal point of ultrasound waves at a particular volume in targeted tissue. However, one skilled in the art will appreciate that some focusing of the ultrasound waves may occur as a function of properties of the tissue through which the ultrasound waves must pass in order to reach targeted tissue. A method and device that provide diffuse ultrasonic waves suitable for use in therapeutic applications are described in U.S. Pat. Nos. 4,501,151 and 4,390,026, the disclosures of which are hereby incorporated herein by reference in their entirety. According to the disclosed method, a diffuse ultrasonic field is produced by frequency modulation of the ultrasonic waves by “white noise”. As a result of variations in acoustic wavelength in this manner, the energy density applied is relatively smoothly distributed throughout the volume of the field, and not concentrated at peaks or absent at nulls that otherwise would be produced by standing waves.

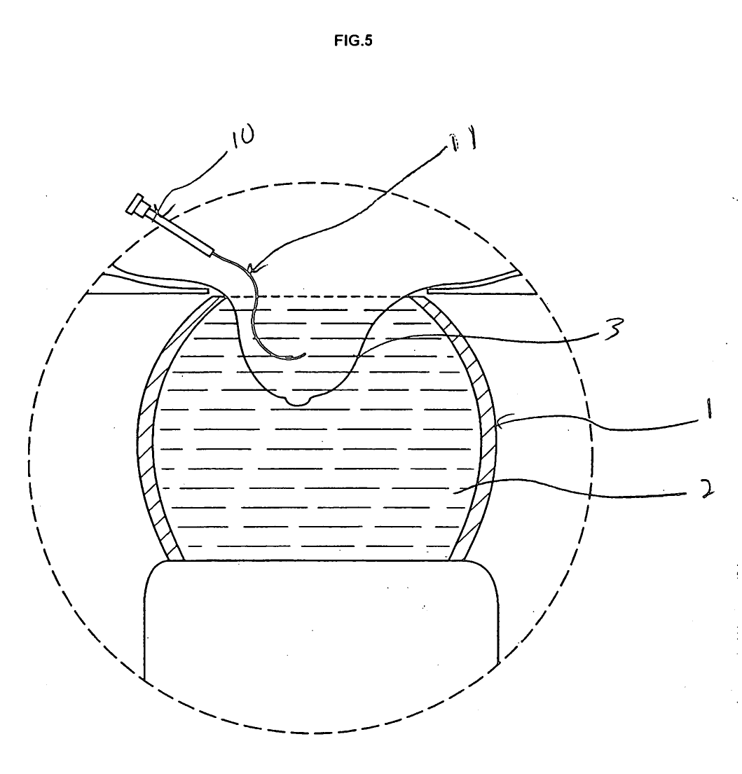

DFHT can be used as a replacement for radiotherapy or in conjunction with radiotherapy, alone or in conjunction with radiotherapy in combination with chemotherapy. As shown in FIG. 5, chemotherapeutic agents can be administered to a patient while the patient is positioned for ultrasound therapy. Syringe 10 having flexible catheter 11 is used to inject an anticancer agent into the breast 3. Concurrently or alternately with the injection, ultrasound is applied through water 2 in tank 1. Anti-cancer drugs for use in chemotherapy are known to those skilled in the art and include TAXOL®, manufactured by Bristol-Myers Squibb; Herceptin® manufactured by Genentech; the combination cytoxan-methotrexate-5-fluorouracil; the combination cytoxan-adriamycin-5-fluorouracil; Ellence® manufactured by Pharmacia & Upjohn; Xeloda® manufactured by Hoffmann-LaRoche, as well as numerous other compounds presently in development or clinical trials. DFHT allows exposure of larger portions of a patient's body, and in particular enables a clinician to treat an entire organ, such as the breast, in contrast with the direct exposure of substantially only the targeted tissue, e.g., tumor, which can be accomplished using conventional focused ultrasound.

“In conjunction with”, as used herein to refer to the combined use of ultrasound-generated hyperthermia and radiation therapy, includes the application of ultrasound simultaneously with radiation, as well as the application of ultrasound sequentially with radiation. “In conjunction with”, as used herein to refer to the combined use of ultrasound-generated hyperthermia and chemotherapy, includes the administration of chemotherapy prior to, during, and/or following the application of ultrasound.

“Simultaneously”, as used herein to refer to the application of ultrasound and radiation to diseased tissue, means that both ultrasound waves and radiation are applied concurrently for at least some period of time. Preferably, ultrasound waves and radiation are applied concurrently for at least about five minutes, more preferably at least about ten minutes, still more preferably for at least about twenty minutes, and even more preferably for at least about 30 minutes. In some embodiments, ultrasound waves and radiation may be applied simultaneously for about forty-five minutes. The practical upper limit of the time of exposure of a patient to ultrasound and/or radiation is determined in substantial part by tolerance on the part of the patient. Simultaneous application of ultrasound waves and radiation is not intended to preclude the application of ultrasound waves or radiation alone for a period of time in addition to the concurrent application thereof. Thus, for example, a treatment modality may include the initial application of radiation alone, followed by the application of ultrasound waves and radiation concurrently, followed by the application of radiation alone. Such alternating treatment may be carried out in any order.

“Sequentially”, as used herein to refer to the alternate application of radiation and ultrasound, means that radiation and ultrasound may be administered in any order and with or without intervening gaps of time, wherein either radiation or ultrasound is applied alone during at least one phase of treatment. The order of application of radiation and ultrasound in sequential application thereof is not believed to be as critical as the use of both forms in conjunction, but may have certain advantages. Optionally, there may be a delay following one or more, or each, application of ultrasound waves and/or each application of radiation. A suitable delay period may be three, four, or five days. The determination of the appropriate delay time between sequential treatments is within the purview of the skilled clinician. While it is not intended that the invention be bound by any particular theory, it is believed that thermal tolerance by targeted and/or surrounding tissue affects the therapeutic effectiveness of hyperthermic treatment. For example, the production of heat shock proteins under hyperthermic conditions has been demonstrated, and it has been suggested that the expression of heat shock proteins plays a role in immune reactions. It is believed that the expression of heat shock proteins following hyperthermic treatment can persist for about 5 or 6 days. In some situations, following a hyperthermic treatment, it may be desirable to withhold a subsequent treatment for a time during which the expression of heat shock proteins subsides, e.g., for about 5, 6 or 7 days. In other situations, it may be desirable to administer a subsequent treatment during a time period in which the expression of heat shock proteins continues, e.g., within about 3 days. For the convenience and tolerance of a patient, treatments may be spaced so that treatments are administered on weekdays and no treatments are administered on weekends. For example, in one preferred embodiment, four treatments may be administered within a two-week period, two treatments being administered per week during weekdays. In another preferred embodiments, two treatments may be administered within a two-week period, one treatment per week during weekdays. Also, in sequential application, radiation is preferably applied for at least about 1 minute, more preferably at least about 1.5 minute. Preferably, ultrasound is applied for at least about ten minutes, more preferably at least about twenty minutes, and still more preferably at least about thirty minutes.

It will be recognized by one skilled in the art that ultrasound is advantageously applied to tissue for a time sufficient for the tissue to reach therapeutic temperature, i.e. to achieve hyperthermia, which can take about 10 or 15 minutes or more. Preferably, the tissue is maintained at therapeutic temperature for at least about 10 minutes, more preferably at least about 20 minutes and more preferably at least about 30 minutes. Also preferably, the tissue is maintained at therapeutic temperature for about 90 minutes or less. The upper limit is not critical; however, generally the relative increase in therapeutic effectiveness with time declines after about one hour of treatment. Typically, total treatment time for ultrasound-induced hyperthermia is about 45 minutes. Generally, relatively higher temperatures can be endured by tissues for relatively shorter times, and a modest temperature elevation may have a substantial effect if sustained over time.

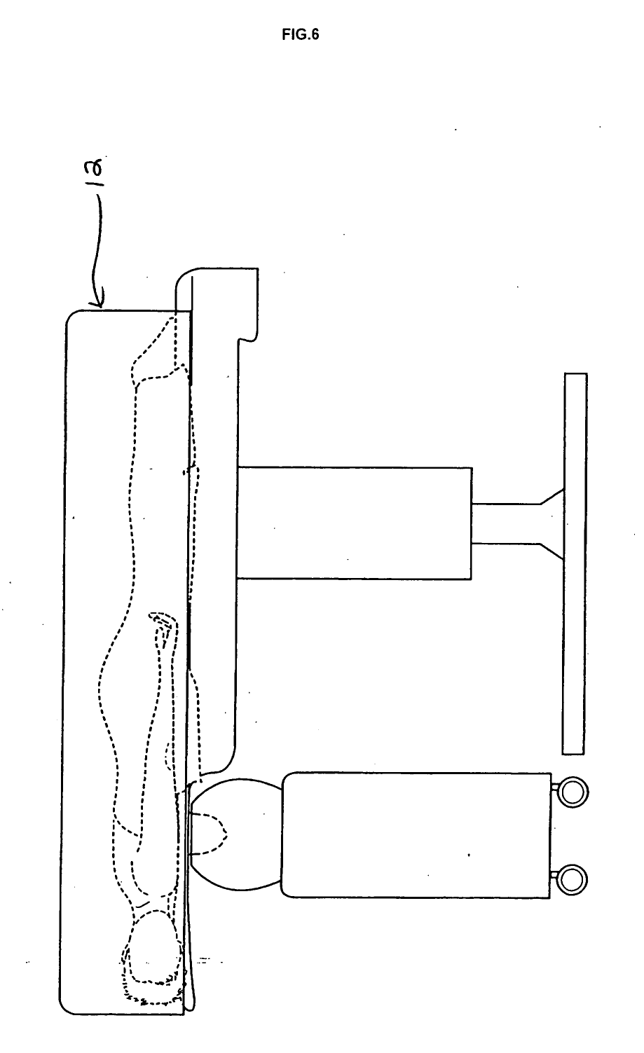

In some embodiments, the application of ultrasound according to the invention may be in conjunction with whole-body hyperthermia, also referred to as “systemic hyperthermia”. With whole-body hyperthermia, substantially the entire body of a patient is maintained at therapeutic temperature, which reduces the effects of phenomena such as perfusion that can reduce local body temperature at targeted tissue and lower the effectiveness of hyperthermic treatment. Whole-body hyperthermia means that a patient's core body temperature is on average at least about 2° C. above the patient's normal body temperature. Whole-body hyperthermia may be achieved by the application of either radiant heat with lamps, or hot water tubes in a hot water vapor saturated atmosphere. As shown in FIG. 6, the patient to be treated with whole-body hyperthermia is placed inside a thermally insulated chamber 12, on biopsy table 4, and ultrasound is administered as described hereinabove. The patient's vital signs are monitored during treatment.

In some embodiments, when radiation is used in conjunction with DFHT in providing treatment to a patient, the treatment is administered according to an exposure scheme referred to herein as “accelerated hyperfractionized dosing”. In accelerated hyperfractionized dosing, two doses of radiation are given per day as opposed to the standard treatment of one dose per day. The second dose is preferably given more than six hours after the first dose, to allow for repair of normal tissue, and for exposure of cells preferably at the most sensitive phase of the cell cycle, particularly G2/M and late G1/early S phases of the cell cycle. Fractionization maximizes the probability that most, preferably all, cells will be in their sensitive phase at the time that one or more doses of radiation, referred to as “fractions”, are delivered. Fractionization is described, for example, in The Physics of Radiotherapy X-Rays from Linear Accelerators, Peter Mecalfe, Tomas Kron and Peter Hoban, Medical Physics Publishing, Madison, Wis., p450, (1997).

The appropriate dosage levels and times for the application of radiation in conjunction with ultrasound-induced hyperthermia can be determined by one skilled in the art, according to accepted clinical practice with regard to radiation therapy. According to presently accepted standards of care, a total dose of, e.g. 50 or 60 Grays, may be fractionated into lower-dose portions that are preferably non-toxic to healthy tissue. Alternatively, a single daily dose of radiation may be administered in conjunction with DFHT. As an example, radiation may be delivered at a level of 1.6 Grays (Gy) initially, either alone or concurrently with ultrasound, and an additional dose of 1.6 Gy may be administered after a suitable delay time, for a total dose of 3.2 Gy per day. Alternatively, a single treatment of preferably not more than 2.8 Gy may be administered per day. In highly preferred embodiments, 1.8 or 2.0 Gy are administered per day. The radiotherapy dose can be varied, e.g., reduced or increased on days when a hyperthermia dose is scheduled. In some embodiments, three treatments utilizing ultrasound, radiation, or both per day can be administered. Such multiple treatments are preferably separated by at least about four hours, more preferably by about six hours, depending in part on the tolerance of the patient. In preferred embodiments, the methods of the invention allow for the reduction of the number and/or severity of treatments.

One method of treating breast cancer according to the invention utilizes radiation of a patient's whole breast, wherein radiation exposure is directed from the side and slightly behind the patient under the patient's armpit in a medial direction through one breast, and from the opposite direction. This method for radiation delivery for modern whole breast treatments is called “opposed tangential fields”. Such medial application of radiation avoids the unnecessary exposure of the patient's heart and/or lung to radiation. Another method utilizes electron beam radiation of lymph nodes around the patient's armpit.

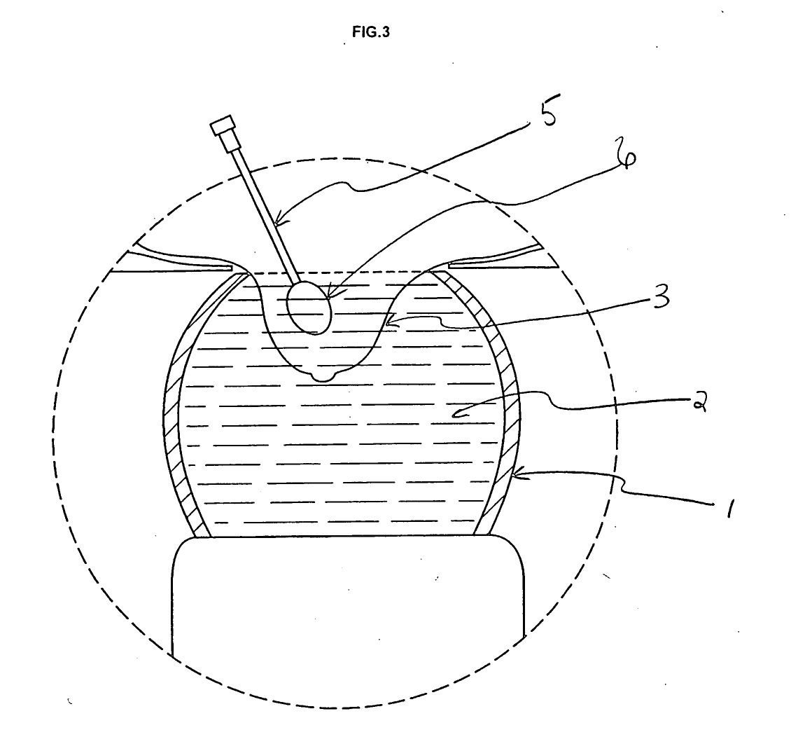

A further method for administering radiation therapy is referred to as “brachytherapy”. In brachytherapy, as illustrated in FIG. 3, a radioactive seed (not shown) is placed at the end of a wire, which is in turn placed into the patient's body with the aid of a balloon catheter 6. Brachytherapy is generally used following removal of a tumor and after healing of the tumor site. The radioactive seed is made of a suitable material, such as Iridium-192.

Still a further method for administering radiation therapy in conjunction with diffuse ultrasound utilizes a mode of radiation administration referred to as a “boost dose”. In a boost dose, radiation is applied either before or after a whole-organ dose and is targeted to the region from which a tumor has been removed. The targeted region is preferably about one and a half times as large (by diameter) as the size of the original tumor. The advisability of the use of a boost dose, and the size of the exposure field, is determined by one skilled in the art based on the region of the body being treated and the size of the tumor or tumors targeted. A boost dose is preferably applied in addition to radiation treatment of a patient's entire organ, such as an entire breast. The level of radiation administered in a boost dose is generally the same as that administered in a daily radiation dose delivered to an entire organ, and may be, for example, 10 Gy administered over 5 days, or 16 Gy administered over 8 days. The methods disclosed herein may be used in combination with or in absence of a boost dose.

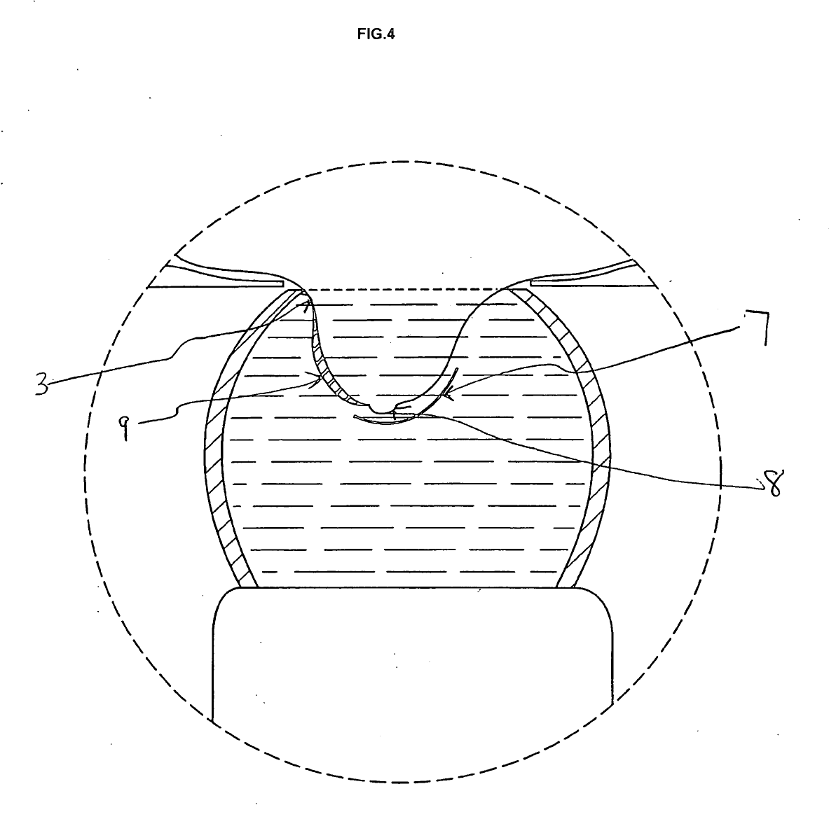

During administration of ultrasound therapy, it may be desirable to utilize a shield to protect the parts of a patient's body that are not intended to be exposed to the ultrasound. For example, for application of ultrasound therapy to breast tissue, a shield that has a contour similar to, or substantially the same as, the contour of the breast may be used. When such a shield is disposed close to but not in contact with the breast, it is referred to as a non-contact shield. An example of a suitable application of a non-contact shield, as illustrated in FIG. 4, is for protection of the nipple while another portion of the breast is being exposed to radiation, to avoid hot spots in the nipple. A non-contact shield 7 is disposed over nipple 8. A non-contact shield is typically made of a radio-opaque metal that reflects ultrasound, or of a material that absorbs ultrasound. Such materials are commercially available and familiar to those skilled in the art. Alternatively, a contact shield, i.e. a shield that is placed in direct contract with the patient's breast or other body part, may be applied. A contact shield may be made of any material that absorbs or reflects ultrasound. The contact shield may be made of a flexible material such as silicone rubber, that has an ultrasound-reflecting material distributed or embedded within it. The shield may have an adhesive material on the outside for affixing the shield to the patient's body. As shown in FIG. 4, shield 9 is affixed to breast 3. The size and position of a contact or non-contact shield depend upon the size and location of the portion of the patient's body that is being treated.

The term “hyperthermia” as used herein to refer to the desired rise in temperature of tissue due to the application of energy such as ultrasound waves, means a therapeutically induced condition in which the temperature of the tissue is detectably higher than the normal range of body temperature in a healthy mammal. Normal ranges of body temperatures vary among different species of mammals and also may vary with the condition of the subject (e.g., by pathogenically induced fever) or the condition of localized tissue (e.g., by local inflammation). Generally, normal body temperature for a human is about 37° C., although individual variations of plus or minus about 2° C. are not uncommon. For therapeutic uses, hyperthermia generally encompasses induced temperature in targeted tissue of a human of at least about 39° C., preferably at least about 40° C., more preferably at least about 41° C., still more preferably at least about 42.5° C., and even more preferably at least about 44° C. It is also preferable to permit a cool down time afterwards before removing the targeted tissue from the treatment apparatus, to reduce the incidence of scalding sensation to the patient. Thus, a patient is preferably allowed to rest in the treatment position following hyperthermic treatment for at least about 5 minutes.

Also preferably, induced body temperature in targeted tissue of a human undergoing hyperthermic therapy does not exceed about 48° C. More preferably, such induced temperature does not exceed about 47° C., and even more preferably, such induced temperature does not exceed about 46° C. It is highly preferred that induced temperature of targeted tissue during hyperthermic therapy is maintained at about 44° C.±2° C. When the temperature of targeted tissue is within the preferred range, there may be local hot and/or cool spots; i.e., local areas having temperatures as low as about 40° C. and as high as about 50° C. It is preferred that such hot and cool spots be minimized. In particular, cool spots may reduce the effectiveness of hyperthermic treatment. In comparison with focused ultrasound, the use of diffuse ultrasound reduces the occurrence of cool spots.

“Radiation”, as used herein to refer to therapeutic energy applied in conjunction with ultrasound, includes all ionizing radiation having energies from about 125 KeV to about 45 MeV, preferably from about 4 MeV to about 23 MeV, more preferably about 8 MeV. Preferred ionizing radiation is photon radiation or electron beam radiation.

In preferred embodiments, ultrasound waves of two or more different frequencies are applied to tissue. The lower the frequency of the ultrasound waves, the more deeply into tissue the ultrasound waves penetrate. Conversely, the higher the frequency of the ultrasound waves, the less deeply into the tissue the ultrasound waves penetrate, and certain higher frequency ultrasound waves do not penetrate substantially beneath the surface of tissue to which the ultrasound waves are applied. If the targeted tissue is immersed in a fluid, higher frequency waves may be substantially absorbed by tissue located at or near the interface between the tissue and the fluid. By controlling the frequency or frequencies of ultrasound waves, a clinician applying the ultrasound waves to tissue to generate hyperthermia can control the temperature and temperature distribution of the tissue. It is thus possible to generate desired temperature profiles throughout the tissue. In some embodiments, it is preferred to generate substantially uniform temperature profiles. By “substantially uniform temperature profiles” is meant a variation in temperature of less than about ±2° C. over a volume of about five hundred cubic centimeters. The use of multiple frequencies also enables a clinician to maintain a therapeutically effective temperature within targeted tissue by continuing the application of ultrasound waves of one frequency while ultrasound waves of a second frequency are turned off in order to obtain measurements of the amount and/or rate of ultrasonic energy being delivered. The use of different frequencies also allows a clinician to alter exposure parameters to account for differences in the size and absorptive properties of both targeted and surrounding tissue, such as, for example, when a patient is having two breasts treated and in particular when the patient's breasts are of different sizes. The presence of subcutaneous fat proximate to targeted tissue may reduce the absorption of ultrasound and thus may complicate achievement of therapeutic temperature. The use of multiple ultrasound frequencies can minimize the effects of anatomical tissue differences.

When two or more frequencies of ultrasound waves are used in the methods described herein, the difference in frequencies is preferably at least about 0.5 MHz, more preferably at least about 0.75 MHz, and even more preferably at least about 1.0 MHz. Also, preferably the lowest frequency of ultrasound waves applied according to the methods described herein is about 0.6 MHz, more preferably at least about 0.7 Hz, even more preferably at least about 0.8 MHz and still more preferably at least about 1.0 MHz. Preferably, the highest frequency of ultrasound waves applied according to the methods described herein is about 8 MHz, more preferably about 7 MHz, even more preferably about 6 MHz, still more preferably about 5 MHz. In certain highly preferred embodiments, the highest frequency of ultrasound waves applied is about 5 MHz. Thus, for example, two frequencies of ultrasound waves, of which the lowest is about 1.0 MHz and the highest is about 2.25 MHz may be applied. In another example, two frequencies of ultrasound waves, of which the lowest is about 1.5 MHz and the highest is about 3.5 MHz may be applied. Optionally, a ultrasound waves of a third frequency, such as about 2.25 MHz may be used in addition to a lower frequency and a higher frequency. The practical upper limit of useful ultrasound frequencies is determined by the absorption of higher frequencies by tissue, i.e., higher frequencies may be absorbed by surrounding tissue, resulting in little or no ultrasound energy reaching targeted tissue. Generally, it is desirable that the ultrasound frequency be 25 MHz or lower. These exemplary frequencies are provided for illustrative purposes only, as the person skilled in the art will be able to determine and apply a preferred combination of ultrasound frequencies for a particular therapy.

Ultrasound waves may be applied to tissue using methods known to those skilled in the art. For example, invasive hyperthermia devices such as implanted transducers and catheters can be used. However, non-invasive techniques for applying ultrasound waves are generally preferred from the standpoint of patient comfort and ease of application. According to the methods described herein, non-invasive methods for administering ultrasound waves are highly preferred. Non-invasive methods for applying ultrasound waves to tissue include the use of an ultrasound transducer immersed in a fluid into which a patient's body, or a portion of a patient's body that encompasses targeted tissue, is also immersed. One apparatus that utilizes such a transducer is described in U.S. Pat. No. 4,936,303, the disclosure of which is hereby incorporated herein by reference in its entirety.

The invention also provides devices for providing ultrasound-generated hyperthermic treatment. The devices include a means for generating unfocused ultrasound waves. Such means are known to those skilled in the art and include, for example, transducers, RF power amplifiers, directional couplers, signal generators, white noise generators and acoustic reflectors. The devices include an means for measuring decay rate. Such means are known to those skilled in the art and include, for example, acoustic hydrophones, high-gain frequency selective amplifiers and transient recorders. Also, the devices include a means for determining the efficiency of the transducer and a means for performing dosimetry. Such means are known to those skilled in the art and include, for example, an acoustic force balance, a calibrated hydrophone, and high-absorbing phantom with embedded thermocouples. Preferably, a device for providing ultrasound-generated hyperthermic treatment includes a means for obtaining data from targeted and/or surrounding tissue in situ. “In situ” means the tissue that is to be treated, in position and prepared for the treatment. The means for obtaining data may include conventional imaging devices, such as MRI scanners. The device also preferably includes minimally invasive thermal probes and a means for inserting and removing such probes. The construction of thermal probes is known to those skilled in the art, and suitable thermal probes include, for example, miniature thermistors and fine-wire thermocouples.

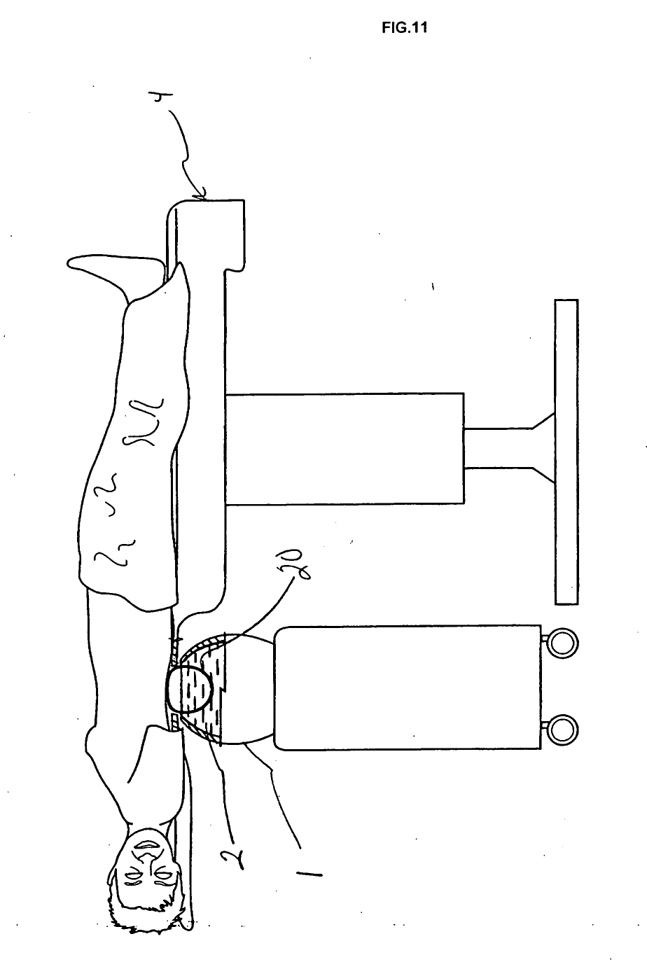

The methods and devices disclosed herein are useful in treating diseased tissue. Diseased tissue includes, in particular, cancerous tissue and more particularly tumors. More particularly, the methods and devices herein are useful in treating cancerous tumors in non-bony tissue, such as the breast, brain, prostate, testicles, kidney, liver, uterus, etc. Such application to diverse tissues is generally illustrated in FIG. 11. Generally, the methods and devices disclosed herein are advantageously used for treatment of cancer in tissues where acoustic impedance differences in targeted and surrounding tissue are minimal. The acoustic impedance for soft, i.e., non-bony, tissue and that of body fluids are similar, and the methods and devices disclosed herein are suitable for use in such soft tissue. In contrast, the presence of bone and/or gas-filled cavities adjacent to soft tissue may reduce the effectiveness of DFHT due in part to the reflection of ultrasound waves. In highly preferred embodiments, the methods and devices disclosed herein are used to treat breast cancer.

In preferred embodiments, the methods and devices disclosed herein incorporate a computer-based controller that can receive data, such as imaging data obtained by ultrasound, computer aided tomography (CT), or magnetic resonance imaging (MRI). Based on the data received, as well as other information such as absorption properties of the targeted and surrounding tissue, ambient temperature, location and distribution of blood vessels in the tissue, flow properties of blood within the blood vessels, the perfusion properties of the blood, heat transfer properties of tissue, density of targeted and surrounding tissue, and sound velocity through the tissue, the controller can determine the appropriate frequencies and exposure times of ultrasound waves to be delivered to the targeted tissue. Preferably, the device includes a computer that can create a three-dimensional model of the targeted and surrounding tissue, prior to, during, and following treatment. The amount of ultrasound energy delivered to the targeted tissue, referred to herein as the “integral dose”, and the time at which such energy is delivered to the tissue, referred to as the “integral dose rate”, can be controlled based on the properties of the tissue and the desired temperature profile. The desired temperature profile can be determined and input by a clinician, and the computer can control the administration of ultrasound waves to achieve the desired temperature profile. In general, for a desired temperature profile, the actual exposure (dose rate and integral dose) varies depending upon the size and properties of the targeted and surrounding tissue.

A central processing unit allows manipulation of the data obtained, and preferably can create a simulated model of the targeted and surrounding tissue including, for example, thermal profiles, density profiles, and dimensions of tissue and blood vessels therein. In addition, it is preferred that the central processing unit can determine from such data the preferred and optimal levels and duration of ultrasound waves to be delivered to the targeted tissue. Thus the duration as well as the associated temperatures maintained can be monitored. This can be advantageous to ensure a particular regimen or perhaps to vary the regimen in an adaptive manner as the treatment progresses. Initially, maintaining and monitoring for particular time and temperature profiles is helpful to permit correlation of treatment conditions and therapeutic results.

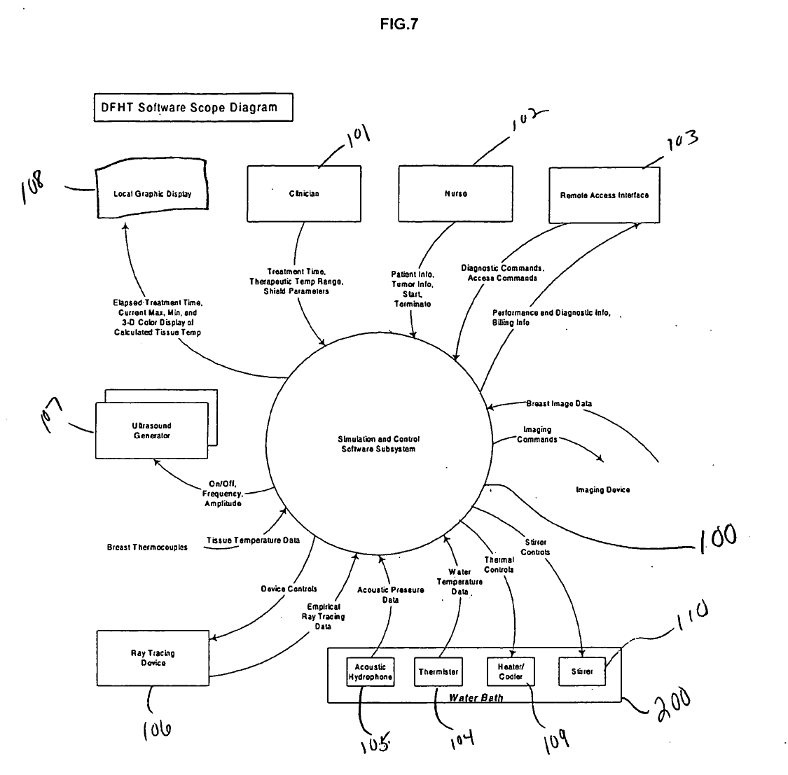

An example of a system for providing DFHT in treatment of breast cancer is illustrated schematically in FIG. 7. According to the embodiment shown, a Simulation and Control Software Subsystem 100 controls the DFHT system of which it is a part, to generate the desired hyperthermia treatment by maintaining the breast tissue within the desired therapeutic range. The Subsystem 100 also can gather performance, diagnostic and billing information, which it provides via a local graphic display and/or remote access. Data is exchanged between Simulation and Control Software Subsystem 100, and a plurality of sources, including a clinician 101, who inputs treatment time, therapeutic temperature range, and shield parameters; nurse 102, who inputs information on the patient such as name and age and on the tumor to be treated, as well as start time and termination time; remote access interface 103, which provides diagnostic commands and access commands; thermistor 104, which inputs water temperature data; acoustic hydrophone 105 which inputs acoustic pressure data; ray tracing device 106, which provides empirical ray tracing data and receives device controls; breast thermocouples, which provide tissue temperature data; heater/cooler 109, which maintains proper temperature; and stirrer 110, which agitates the water in water bath 200, in which breast (not shown) is located for treatment. In addition, the Simulation and Control Software Subsystem provides on/off, frequency and amplitude instructions to ultrasound generator, and provides elapsed treatment time and optionally a three-dimensional color display of the calculated tissue temperature to Local Graphic Display.

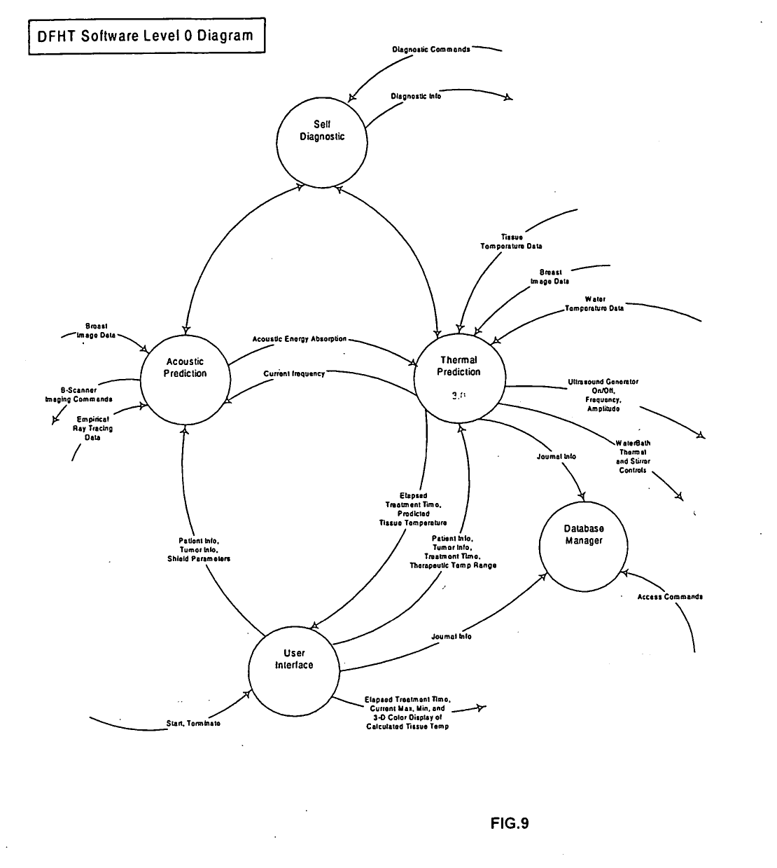

In FIG. 9 is shown schematically a control and feedback system for use in accordance with some embodiments of the invention. The system allows for input of data at a user interface, such as patient information, treatment time, therapeutic temperature range, properties of any shield to be used, and information about the tumor to be treated. Journal information can be input into a database manager for record keeping. Journal information can include treatment parameters and results, and results of thermal or acoustic prediction. A program for acoustic prediction receives empirical data including breast image data and empirical ray tracing data. A program for thermal prediction receives tissue temperature data, breast image data, water temperature data, patient information, tumor information, treatment time, and therapeutic temperature range. Exemplary parameters that can be input for thermal prediction or acoustic prediction are shown in Table 1.

TABLE I
Typical Values for the Acoustic and
Thermodynamic Parameters of Tissue.

| Parameter | Fat | Soft-Tissue | Muscle |
|---|---|---|---|
| Density (g/cm³) | 0.97 | 1.04 | 1.07 |
| Velocity (cm/sec) × 10⁵ | 1.44 | 1.52 | 1.57 |
| Absorption (Np/cm) | 0.05 | 0.10 | 0.13 |

Additional tissue parameters that can be input, which is available in reference publications available to those skilled in the art, includes specific heat at constant pressure, and thermal conductivity. Optionally, a self-diagnostic program can be included, which relays diagnostic information in response to commands.

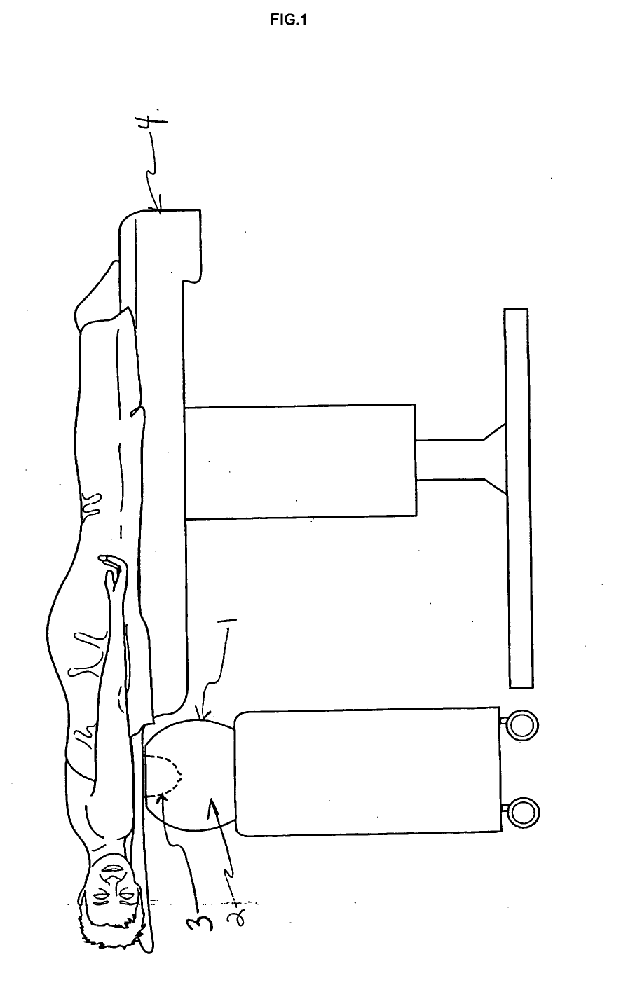

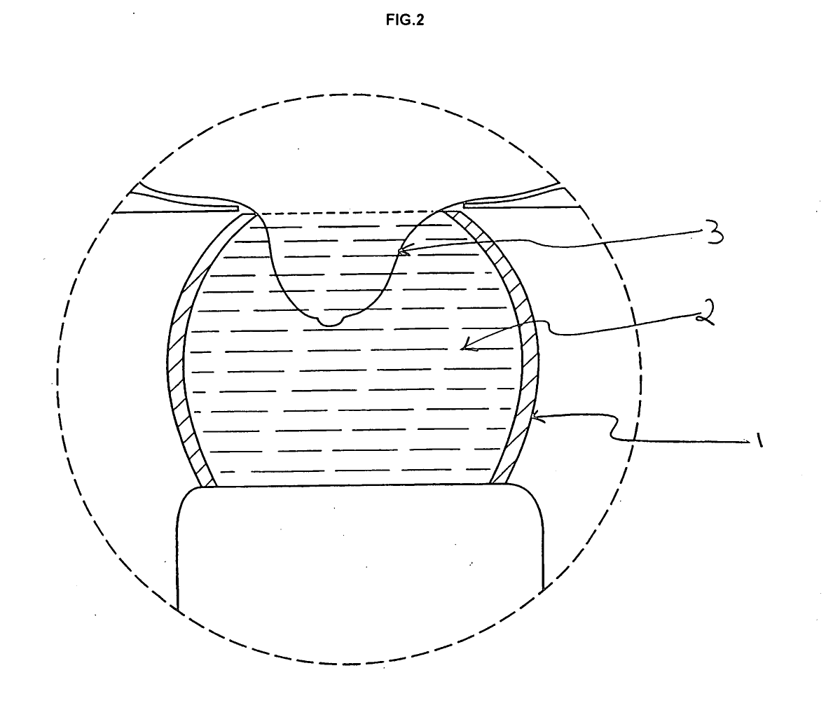

Hyperthermic treatment by the delivery of ultrasound waves to targeted tissue can be achieved in an exposure tank. An exemplary embodiment of a device including such an exposure tank is shown in FIGS. 1 and 2. In the exemplified embodiment, a tank 1 contains water 2. A patient rests on biopsy table 4 and the patient's breast 3 is disposed within the water 2. Ultrasound waves travel unfocused through the water and the breast 3. The device includes a computer, which runs a computer algorithm that allows simulation by the computer of the structure and physiological characteristics of the breast.

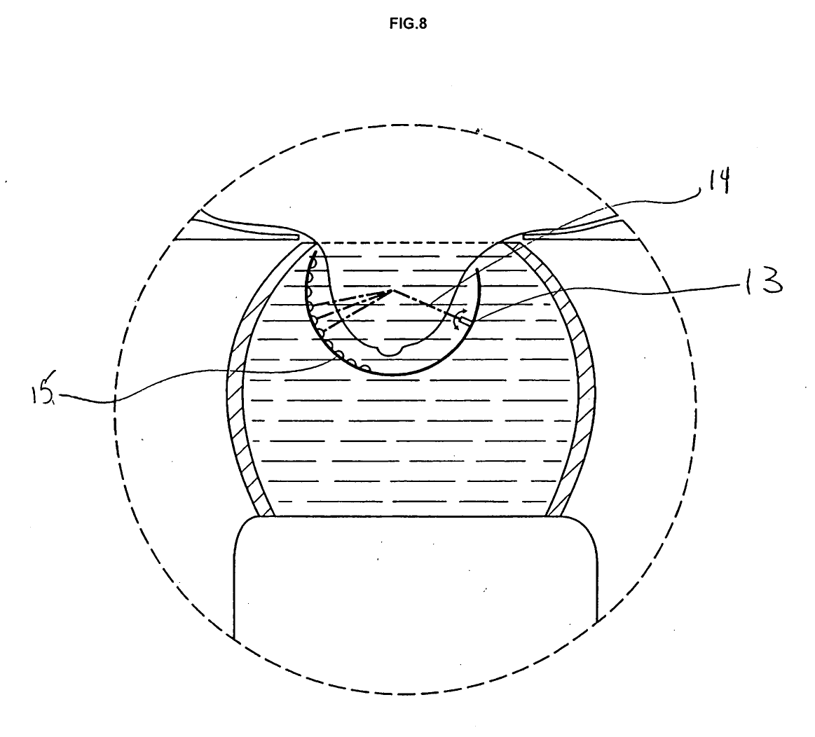

If desired, as an alternative or supplement to simulation, the computer is and appropriate detectors can measure characteristics of the tissue empirically. An empirical method for determining the characteristics of breast tissue is illustrated schematically in FIG. 8. A transducer 13 can be used to produce an acoustic beam 14 having a predetermined amplitude and direction. An array of transducers 15 on opposite sides of the breast records the direction and amplitude of ultrasound waves. As a simplification, reflection of acoustic waves can be ignored in the computer algorithm or when using the empirical method. This method can be used in combination with, or as a substitute for, an acoustic simulation model.

In preferred embodiments, an isolated chamber can be contained within the exposure tank. That portion of a patient's body in which the targeted tissue is located is encased within the isolated chamber. The isolated chamber can be made from any material that is at least partially transparent, and preferably substantially transparent, to ultrasound waves at the frequency being applied. The material can be entirely transparent, or transparent only where the material contacts the ultrasound-transmitting device, e.g., transducer, to allow the ultrasound waves to pass through the material to contact the targeted tissue.

An example of such an isolated chamber is a bag made of a polymeric material such as, for example, polyester film, or film made of polyethylene or other polyolefin. Any material that is substantially transparent to acoustic waves is suitable for forming a bag. Preferably, the material used to form a bag has substantially the same density as the surface with which the bag is to be in contact, e.g., human skin. While physical properties such as break strength are less critical than transparency to ultrasound waves, it is highly preferred that the material of which the isolated chamber is made be sufficiently strong to withstand use during at least one complete treatment session without bursting or tearing. Polyester films have been found to be particularly suitable. In referring to embodiments in which a polyester or other polymeric film is used for the isolated chamber, the chamber may be referred to as a “bag” herein for simplicity. A bag can offer the advantage of disposability, thereby reducing the need for sterilization between patients.

When an isolated chamber is used, the isolated chamber is filled with the same fluid that fills an exposure tank that surrounds the isolated chamber. The exposure tank may be of any shape, such as, for example, a rectangular solid having four side walls and a bottom wall. In some embodiments, an isolated chamber may be used in the absence of an exposure tank. In other embodiments, an isolated chamber having an outer surface may be placed within an exposure tank so that the outer surface of the isolated chamber is at least partially, and preferably substantially entirely, in contact with the exposure tank. Whether or not an exposure tank is used, the bag is in contact with one or more ultrasound transducers mounted externally to the bag, either directly or through a wall of the exposure tank. Preferably neither the bag nor the exposure tank significantly absorbs ultrasound at the frequency that is to be applied during treatment. Preferably, a material such as an ultrasound “coupling gel”, known to those skilled in the art, is applied to the surface of the bag and/or transducer. An exemplary embodiment in which an isolated chamber is used is illustrated in FIG. 11. Patient is supine on biopsy table 4. Tank 1 contains water 2 and isolated chamber 20 in contact with patient's back adjacent to kidney (not shown).

Optionally, a device as disclosed herein may include a means for degassing the fluid contained within the exposure tank and/or bag. Such means may be, for example, a vacuum source in fluid contact with the fluid contained within the exposure tank and/or bag, so that vacuum may be applied to the fluid to remove one or more gases that may be in the fluid. Examples of gases that may be in the fluid and are which are preferably removed prior to and/or during treatment are carbon dioxide, air, oxygen and nitrogen.

Optionally, a device may include one or more pairs of inlet and outlet ports. Such inlet and outlet ports allow the removal of fluid from the exposure tank and/or bag, either temporarily for alteration, or permanently for replacement. Such alteration may include, for example, heating, cooling, or degassing. Degassing of the fluid following removal eliminates the need to apply vacuum directly to the exposure tank.

### EXAMPLE 1

### Clinical Application to the Treatment of a Patient Diagnosed with Breast Cancer Using DFHT and Radiation

After a suitable workup and review of patient records and treatment plan, the patient receives an accelerated hyperfractionized dose of radiation from a linear accelerator (dose 1.6 Gy, exposure time 1.5 minutes, ionizing energy 6 MeV). Within 30 minutes of the ionizing exposure, the patient receives DFHT, which provides substantially uniform temperature profiles of 44.0±2 degrees C. within the entire breast being treated, for at least 30 minutes.

On the same day, after receiving the DFHT treatment and after a delay of at least six hours, the patient returns to the radiation treatment room and receives a second radiation treatment. The patient returns the next day and the treatment is repeated, this time without the hyperthermia treatment. The treatment is repeated 5 days per week for two weeks, for a total of four hyperthermia treatments and 20 radiotherapy treatments. The total dose of radiation is 32 Gy, which is about 40% less than the dose typically provided in conventional radiotherapy.

### EXAMPLE 2

### Comparative Example—Treatment of Cancer Using Focused Ultrasound

After confirmation of the diagnosis and while waiting a date for surgery, the patent receives one or more treatments of focused ultrasound directed at the tumor. The ultrasound is provided by, for example, a SONOTHERM 1000 manufactured by Labthermics. After application of ultrasound and the resulting heating, the tumor shrinks, facilitating the surgical removal of the tumor. After surgery and a sufficient time for recovery, the patient may undergo conventional chemotherapy. The tumor cells may become hypoxic if the tumor has outgrown its blood supply. Focused ultrasound is particularly effective for treating hypoxic cells, while other treatment modalities may fail. Since the production of free radicals is less likely in hypoxic cells than in well oxygenated cells, and free radicals are produced when radiation interacts with water, and particularly oxygen atoms, hypoxic cells are particularly resistant to injury from radiation. Since blood flow is restricted in hypoxic cells, such cells may also be resistant to chemotherapy, since they are likely to receive a lower local or internal dose of the drug. After surgery, the patient may undergo conventional chemotherapy.

### EXAMPLE 3

### Clinical Application to the Treatment of a Patient Diagnosed with Breast Cancer Using DFHT and Chemotherapy

After a suitable workup and review of patient records and treatment plan, the patient receives DFHT, which provides substantially uniform temperature profiles of between about 42° C. and 46° C. within the entire breast being treated, for at least about 10 minutes, preferably for about 30 minutes. The effectiveness of DFHT treatment is expected to increase with treatment time, but such increase is minimized after treatment times exceeding 45 minutes. The patient returns on the third day following treatment, and the treatment is repeated. The treatment is repeated 2 days per week for two weeks, for a total of 4 hyperthermia treatments.

In addition to the DFHT, on each day of treatment, the patient is given a one-hour intraperitoneal infusion of the anti-cancer drug Herceptin® into the breast during the hyperthermia treatment and while the patient lays prone on the biopsy table. At the end of the hyperthermia treatment but before the patient sits up, the intraperitoneal cavity of the breast is flushed with saline to remove the anti-cancer drug. This procedure is called “targeted chemotherapy using hyperthermia”, and greatly reduces the patients systemic side effects as compared to conventional chemotherapy.

A number of additional applications and variations also are possible, within the scope of the invention as defined in the appended claims.

1. A method for treating diseased tissue in a patient, comprising inducing in said tissue regional hyperthermia by applying unfocused ultrasound waves to said tissue.

2. A method of claim 1, further comprising applying to said tissue high-energy radiation.

3. A method of claim 2, wherein said ultrasound waves and said high-energy radiation are applied simultaneously.

4. A method of claim 2, wherein said ultrasound waves and said high-energy radiation are applied sequentially.

5. A method of claim 4, wherein said diffuse ultrasound waves and radiation are applied according to a treatment schedule in which radiation is administered for from about 1 minute to about two minutes, and after a delay of at least about 30 minutes, diffuse ultrasound is applied for at least about ten minutes.

6. A method of claim 5, wherein said radiation is administered for 1.5 minute, said delay is about 30 minutes, and said diffuse ultrasound is applied such that the temperature of the diseased tissue is maintained at 42° C. or higher for at least about 10 minutes.

7. A method of claim 6 wherein said temperature of the diseased tissue is maintained within the range of about 42° C. to about 46° C. for at least about 10 minutes.

8. A method of claim 5 wherein at least one of said administration of said radiation for 1.5 minute and said application of diffuse ultrasound such that the temperature of the diseased tissue is maintained at 42° C. or higher is repeated.

9. A method of claim 5 wherein said treatment schedule is repeated within not less than 3 days.

10. A method of claim 9 wherein said treatment schedule is repeated within not less than one week.

11. A method of claim 1, further comprising administering to said patient one or more pharmacological agents.

12. A method of claim 5, wherein said pharmacological agent is selected from the group consisting of TAXOL®, Herceptin, cytoxan, methotrexate, 5-fluorouracil, adriamycin, Ellence®, and Xeloda.

13. A method of claim 1, wherein said ultrasound waves have a frequency from about 0.6 MHz to about 8 MHz.

14. A method of claim 1 wherein said patient is a human.

15. A method of claim 1 wherein said diseased tissue comprises one or more microtumors.

16. A method of claim 1 wherein said diseased tissue comprises breast cancer.

17. A method of claim 1, wherein said application of ultrasound waves is computer controlled.

18. A method of claim 17, wherein said computer controlling comprises obtaining data from said tissue, processing said data, and controlling at least one of the duration and frequency of ultrasound waves applied to said tissue.

19. A device for providing ultrasound-generated hyperthermic treatment to a patient having diseased tissue in need of treatment, comprising:
a means for generating unfocused ultrasound waves;
a means for obtaining data from said tissue in situ;
a central processing unit;
at least one of an exposure tank and an isolated chamber; and
a means for directing said ultrasound waves to said tissue.
